SEQUENCE LISTING (3 sequences)

SEQ ID NO: 1 - protein, 394 aa, Homo sapiens
MPVPWFLLSLALGRSPVVLSLERLVGPQDATHCSPGLSCRLWDSDILCLPGDIVPAPGPVLAPTHLQTELVLRCQKETDCDLCLRVAVHLAVHGHWEEPEDEEKFGGAADSGVEEPRNASLQAQVVLSFQAYPTARCVLLEVQVPAALVQFGQSVGSVVYDCFEAALGSEVRIWSYTQPRYEKELNHTQQLPALPWLNVSADGDNVHLVLNVSEEQHFGLSLYWNQVQGPPKPRWHKNLTGPQIITLNHTDLVPCLCIQVWPLEPDSVRTNICPFREDPRAHQNLWQAARLRLLTLQSWLLDAPCSLPAEAALCWRAPGGDPCQPLVPPLSWENVTVDKVLEFPLLKGHPNLCVQVNSSEKLQLQECLWAEGSSPWRVLTTRPAVRPVSAAMGR

SEQ ID NO: 2 - DNA, 1185 bp, Homo sapiens
atgcctgtgccctggttcttgctgtccttggcactgggccgaagcccagtggtcctttctctggagaggcttgtggggcctcaggacgctacccactgctctccgggcctctcctgccgcctctgggacagtgacatactctgcctgcctggggacatcgtgcctgctccgggccccgtgctggcgcctacgcacctgcagacagagctggtgctgaggtgccagaaggagactgactgtgacctctgtctgcgtgtggctgtccacttggccgtgcatgggcactgggaagagcctgaagatgaggaaaagtttggaggagcagctgactcaggggtggaggagcctaggaatgcctctctccaggcccaagtcgtgctctccttccaggcctaccctactgcccgctgcgtcctgctggaggtgcaagtgcctgctgcccttgtgcagtttggtcagtctgtgggctctgtggtatatgactgcttcgaggctgccctagggagtgaggtacgaatctggtcctatactcagcccaggtacgagaaggaactcaaccacacacagcagctgcctgccctgccctggctcaacgtgtcagcagatggtgacaacgtgcatctggttctgaatgtctctgaggagcagcacttcggcctctccctgtactggaatcaggtccagggccccccaaaaccccggtggcacaaaaacctgactggaccgcagatcattaccttgaaccacacagacctggttccctgcctctgtattcaggtgtggcctctggaacctgactccgttaggacgaacatctgccccttcagggaggacccccgcgcacaccagaacctctggcaagccgcccgactgcgactgctgaccctgcagagctggctgctggacgcaccgtgctcgctgcccgcagaagcggcactgtgctggcgggctccgggtggggacccctgccagccactggtcccaccgctttcctgggagaacgtcactgtggacaaggttctcgagttcccattgctgaaaggccaccctaacctctgtgttcaggtgaacagctcggagaagctgcagctgcaggagtgcttgtgggctgagggcagctcgccttggagagtacttactacaagacctgcagtcaggccagtgtctgcagctatgggacgatga

SEQ ID NO: 3 - protein, 409 aa, Homo sapiens
MPVPWFLLSLALGRSPVVLSLERLVGPQDATHCSPGLSCRLWDSDILCLPGDIVPAPGPVLAPTHLQTELVLRCQKETDCDLCLRVAVHLAVHGHWEEPEDEEKFGGAADSGVEEPRNASLQAQVVLSFQAYPTARCVLLEVQVPAALVQFGQSVGSVVYDCFEAALGSEVRIWSYTQPRYEKELNHTQQLPDCRGLEVWNSIPSCWALPWLNVSADGDNVHLVLNVSEEQHFGLSLYWNQVQGPPKPRWHKNLTGPQIITLNHTDLVPCLCIQVWPLEPDSVRTNICPFREDPRAHQNLWQAARLRLLTLQSWLLDAPCSLPAEAALCWRAPGGDPCQPLVPPLSWENVTVDKVLEFPLLKGHPNLCVQVNSSEKLQLQECLWAEGSSPWRVLTTRPAVRPVSAAMGR

(19) United States
(12) Patent Application Publication
(10) Pub. No.: US 2008/0305078 A1
(43) Pub. Date: Dec. 11, 2008

(54) Soluble Il-17Rc Variant and Uses Thereof

(75) Inventors: Laurent Magnenat, Geneve (CH); Christine Power, Thoiry (FR)

Correspondence Address: SALIWANCHIK LLOYD & SALIWANCHIK;A PROFESSIONAL ASSOCIATION, PO BOX 142950, GAINESVILLE, FL, 32614-2950, US

(73) Assignee: LABORATOIRES SERONO SA, Coinsins, Vaud (CH)

(21) Appl. No.: 12/158,572

(22) PCT Filed: Dec. 22, 2006
(86) PCT No.: PCT/EP2006/012467

§ 371 Date: Aug. 7, 2008

Related U.S. Application Data
(60) Provisional application No. 60/759,449, filed on Jan. 17, 2006.

(30) Foreign Application Priority Data
Dec. 22, 2005 (EP) 05112854.4

(51) Int. Cl. A61K 38/20; C07K 14/54; C12N 15/24; C12P 21/02; A61P 29/00; A61P 37/00; C12N 5/10; C12N 15/85; C07K 16/24

(52) U.S. Cl. 424/85.2; 530/351; 536/23.5; 435/320.1; 530/387.9; 435/69.52; 435/325

(57) ABSTRACT

The present invention relates to a new soluble IL-17RC variant and its therapeutic uses thereof, in particular for treating or preventing inflammatory or autoimmune disorders or neurological diseases.

### FIELD OF THE INVENTION

The present invention relates to an IL-17RC soluble variant polypeptide, sIL-17RC, and a nucleic acid molecule encoding the same. The invention also relates to the use of the new sIL-17RC variant in preventing and/or treating inflammatory, autoimmune or neurological disorders.

### BACKGROUND OF THE INVENTION

IL-17A is a major proinflammatory cytokine secreted by activated T-lymphocytes and it is the founding member of the IL-17 cytokine family. The other family members are IL-17B (also known as chondroleukin), IL-17C, IL-17E and IL-17F. The receptors for the IL-17 family members are IL-17R, IL-17RB (IL-17RH1), IL-17RC (IL-17RL), IL-17RD (hSEF) and IL-17RE. However the specificities of many of these receptors have not yet been established and not much is known about their functions or signalling pathways. The IL-17 cytokine family and receptors have been reviewed by Moseley et al. (Moseley et al., 2003).

IL-17R, the originally described IL-17R receptor is a type I transmembrane protein consisting of 293 aa extracellular domain, a 21 amino acid transmembrane domain and a long 525 amino acid cytoplasmic tail (Yao et al., 1997). Its mRNA was shown to be extensively expressed in the lungs, kidneys, liver and spleen as well as isolated fibroblasts, epithelial cells, mesothelial cells and various myeloid cells from rats and mice (Yao et al., 1997). IL-17R binds IL-17A with a relatively low affinity (Yao et al., 1997). Because IL-17F has some homology with IL-17A (58% at the protein level) it may use IL-17R for signalling (Hymowitz et al., 2001).

IL-17RB (IL-17RH1) is expressed in human kidney, pancreas, liver, brain and intestines and serves as a receptor for IL-17B and IL-17E (Lee et al., 2001; Shi et al., 2000). The alternative splicing of IL-17RB results in secreted soluble proteins (Tian et al., 2000).

Haudenschild et al., (Haudenschild et al., 2002) cloned and characterized a single-pass transmembrane protein with limited homology to IL-17R that was named IL-17-RL (receptor-like). This receptor is also known as IL-17RC or Zcytor14 in WO01/04304. 11 splice variants of IL-17 RC (IL-17 RL) transcribed from 19 exons were also reported. The alternative splicing of the RNA was shown to introduce frameshifts and stop codons before the C-terminal transmembrane domain resulting in the translation of secreted rather than transmembrane protein. These findings were also disclosed in WO 02/38764.

The presence of soluble IL-17RB and IL-17RC decoy receptors and the tissue specific regulation of IL-17-RC mRNA splicing to generate different receptor isoforms, hint that the regulation of IL-17 pathways is complex and tightly regulated (Moseley et al., 2003).

IL-17 RD also exhibits alternative splicing (Moseley et al., 2003). Finally IL-17RE is expressed in human brain, prostate and pancreas. The ligands to IL-17RC, IL-17RD and IL-17RE remain uncertain.

IL-17 family members play an active role in inflammatory disease, autoimmune disease and cancer (reviews by Kolls and Linden, 2004; Moseley et al., 2003). IL-17 contributes to the pathogenesis of arthritis (Lubberts, 2003). Neutralizing endogenous IL-17 during reactivation of antigen-induced arthritis was shown to prevent joint inflammation and bone erosion (Koenders et al., 2005).

The contribution of other IL-17-related molecules and their receptors to rheumatoid arthritis (RA) pathogenesis was assessed by Hwang S Y and Kim H Y. Their data suggest that IL-17 homologs homologs should also be considered as targets for immune modulation in the treatment of RA joint inflammation (Hwang and Kim, 2005).

IL-17 family members may exert both pro- or antitumor effects, depending on the immuogenicity of the tumor, the immune status of the host, and the angiogenic activity of IL-17 family member.

IL-17RC could play a role in osteoarthritis (OA) as it was isolated form an expression cloning cDNA library from OA cartilage. Using a functional assay screening method, it showed induction of chondrocyte cluster formation, a phenotype associated with OA (Quintavalla et al., 2005). Soluble sIL-17RC isoform such as presented here may antagonize IL-17RC effect in this disease.

In addition, soluble IL-17RC may have a role in cancer progression, such as prostate cancer where IL-17RC distribution levels in stroma correlate with grades of cancer. Thus, IL-17RC was proposed to be a diagnostic marker in determining the aggressiveness of prostate cancers (Haudenshild et al. 2002; WO 02/38764).

WO 2005/051422, provides a method for the treatment and/or prophylaxis of multiple sclerosis (MS) comprising administering a therapeutically effective amount of an inhibitor of IL-17 activity.

Also, antagonists to IL-17 cytokines, particularly IL-17B (chondroleukin), is involved in catabolic degradation of bone and cartilage. Thus, IL-17 RC binding to IL-17B was proposed to provide methods to reduce or ameliorate disease or injury of bone and cartilage by inhibiting chondroleukin activity by using soluble or immobilized IL-17 RC (WO 02/38764).

The discovery and development of new soluble proteins and their receptors, including ones similar to cytokines, should therefore contribute to new therapies for a wide range of inflammatory, autoimmune and neurological conditions.

### SUMMARY OF THE INVENTION

The present invention provides a new soluble IL-17RC variant polypeptide having the sequence of SEQ ID No. 1, polynucleotides encoding the novel protein, and uses thereof.

### BRIEF DESCRIPTION OF THE DRAWINGS

FIG. 1: shows an alignment of sIL-17RC cDNA, protein and primer sequences.

FIG. 2: Secretion of sIL-17RC and control sIL-17RCexo7 isoforms from HEK293 cells transfected with pEAK12d and pDEST12.2 expression vectors.

### DETAILED DESCRIPTION OF THE INVENTION

This invention describes a novel soluble isoform of the IL-17RC receptor (sIL-17RC).

Therefore, a first aspect of the invention relates to a new soluble IL-17RC variant polypeptide comprising the amino acid sequence of SEQ ID NO: 1.

The terms “soluble IL-17RC variant”, “sIL-17RC” or “IL-17RC_I22” as used herein, relates to a soluble/secreted form of the human IL-17RC receptor. The amino acid sequence of sIL-17RC is reported herein as SEQ ID NO: 1 of the annexed sequence listing.

The polypeptide of the invention contains most of the reported extracellular domain of IL-17RC receptor (NCBI protein entry NP_{—}703190: interleukin 17 receptor C isoform 2 precursor), except exons 7 and 14 which are spliced out, and it terminates with a non-membrane spanning C-terminal exon 15, which introduces a frameshift and a stop codon before the position of the reported IL-17RC transmembrane domain.

In a preferred embodiment of the invention, sIL-17RC is fused to a carrier molecule, a peptide or a protein that promotes the crossing of the blood brain barrier (“BBB”). This serves for proper targeting of the molecule to the site of action in those cases, in which the CNS is involved in the disease. Modalities for drug delivery through the BBB entail disruption of the BBB, either by osmotic means or biochemically by the use of vasoactive substances such as bradykinin. Other strategies to go through the BBB may entail the use of endogenous transport systems, including carrier-mediated transporters such as glucose and amino acid carriers; receptor-mediated transcytosis for insulin or transferrin; and active efflux transporters such as p-glycoprotein; Penetratin, a 16-mer peptide (pAntp) derived from the third helix domain of Antennapedia homeoprotein, and its derivatives. Strategies for drug delivery behind the BBB further include intracerebral implantation.

The proteins according to the present invention may be glycosylated or non-glycosylated, they may be derived from natural sources, such as body fluids, or they may preferably be produced recombinantly. Recombinant expression may be carried out in prokaryotic expression systems such as E. coli, or in eukaryotic, such as insect cells, and preferably in mammalian expression systems, such as CHO-cells or HEK-cells.

“Functional derivatives” as used herein, cover derivatives of sIL-17RC, and their muteins and fused proteins, which may be prepared from the functional groups which occur as side chains on the residues or the N- or C-terminal groups, by means known in the art, and are included in the invention as long as they remain pharmaceutically acceptable, i.e. they do not destroy the activity of the protein which is substantially similar to the activity of sIL-17RC, and do not confer toxic properties on compositions containing it.

These derivatives may, for example, include polyethylene glycol side-chains, which may mask antigenic sites and extend the residence of a sIL-17RC in body fluids. Other derivatives include aliphatic esters of the carboxyl groups, amides of the carboxyl groups by reaction with ammonia or with primary or secondary amines, N-acyl derivatives of free amino groups of the amino acid residues formed with acyl moieties (e.g alkanoyl or carbocyclic aroyl groups) or O-acyl derivatives of free hydroxyl groups (for example that of seryl or threonyl residues) formed with acyl moieties.

Therefore, in a preferred embodiment of the present invention, soluble IL-17RC variant is PEGylated.

Functional derivatives of sIL-17RC may be conjugated to polymers in order to improve the properties of the protein, such as the stability, half-life, bioavailability, tolerance by the human body, or immunogenicity. To achieve this goal, sIL-17RC may be linked e.g. to Polyethlyenglycol (PEG). PEGylation may be carried out by known methods, described in WO 92/13095, for example.

Proteins according to the present invention may be fused with another protein, polypeptide or the like which, e.g. has an extended residence time in body fluids. In a further preferred embodiment of the invention A sIL-17RC may thus be fused to, e.g. an immunoglobulin or a fragment thereof. The fusion may be direct, or via a short linker peptide which can be as short as 1 to 3 amino acid residues in length or longer, for example, 13 amino acid residues in length. Said linker may be a tripeptide of the sequence E-F-M (Glu-Phe-Met), for example, or a 13-amino acid linker sequence comprising Glu-Phe-Gly-Ala-Gly-Leu-Val-Leu-Gly-Gly-Gln-Phe-Met introduced between sIL-17RC sequence and the immunoglobulin sequence, for instance. The resulting fusion protein has improved properties, such as an extended residence time in body fluids (half-life), or an increased specific activity, increased expression level. The Ig fusion may also facilitate purification of the fused protein.

In a yet another preferred embodiment, sIL-17RC is fused to the constant region of an Ig molecule. Preferably, it is fused to heavy chain regions, like the CH2 and CH3 domains of human IgG1, for example. Other isoforms of Ig molecules are also suitable for the generation of fusion proteins according to the present invention, such as isoforms IgG2 or IgG4, or other Ig classes, like IgM, for example. Fusion proteins may be monomeric or multimeric, hetero- or homomultimeric. The immunoglobulin portion of the fused protein may be further modified in a way as to not activate complement binding or the complement cascade or bind to Fc-receptors.

Further fusion proteins of sIL-17RC may be prepared by fusing domains isolated from other proteins allowing the formation or dimers, trimers, etc. Examples for protein sequences allowing the multimerization of the polypeptides of the Invention are domains isolated from proteins such as hCG (WO 97/30161), collagen X (WO 04/33486), C4BP (WO 04/20639), Erb proteins (WO 98/02540), or coiled coil peptides (WO 01/00814).

In another embodiment of the invention, the soluble IL-17RC variant polypeptide is encoded by a nucleic acid molecule, preferably a nucleic acid molecule comprising the cDNA of SEQ ID No. 2 of the annexed sequence listing.

A further embodiment of the invention relates to a vector comprising nucleic acid molecules encoding soluble IL-17RC variant.

The term “vector” refers to any carrier of exogenous DNA or RNA that is useful for transferring exogenous DNA to a host cell for replication and/or appropriate expression of the exogenous DNA by the host cell, such as e.g. plasmids, expression vectors, viral vectors etc.

Expression vector sequences are well known in the art, they comprise further elements serving for expression of the gene of interest. They may comprise regulatory sequence, such as promoter and enhancer sequences, selection marker sequences, origins of multiplication, and the like.

A gene therapeutic approach may also be used for treating and/or preventing the disease. Advantageously, the expression of sIL-17RC will then be in situ.

In a preferred embodiment, the expression vector is a lentiviral derived vector. Lentiviral vectors have been shown to be very efficient in the transfer of genes, in particular within the CNS. Other well established viral vectors, such as adenoviral derived vectors, may also be used according to the invention.

A targeted vector may be used in order to enhance the passage of sIL-17RC across the blood-brain barrier. Such vectors may target for example the transferrin receptor or other endothelial transport mechanisms.

The use of a vector for inducing and/or enhancing the endogenous production of sIL-17RC in a cell normally silent for expression of sIL-17RC, or expressing amounts of sIL-17RC which are not sufficient, are also contemplated according to the invention. The vector may comprise regulatory sequences functional in the cells desired to express sIL-17RC. Such regulatory sequences may be promoters or enhancers, for example. The regulatory sequence may then be introduced into the appropriate locus of the genome by homologous recombination, thus operably linking the regulatory sequence with the gene, the expression of which is required to be induced or enhanced. The technology is usually referred to as “endogenous gene activation” (EGA), and it is described e.g. in WO 91/09955.

In a preferred embodiment of the invention, the expression vector may be administered by intramuscular injection.

The invention further relates to a host cell transfected with the vectors of the invention. Many host cells are suitable in accordance with the present invention, such as primary or established cell lines from a wide variety of eukaryotes including plant and animal cells, exemplified by CHO, BHK, HEK293 or other immortalized and/or transformed cells.

In another aspect, the invention relates to a process for the production of a soluble IL-17RC variant polypeptide comprising the step of culturing a host cell transfected with at least one vector according to the invention.

In a further preferred embodiment, the process further comprises the step of isolating the expression proteins from the host cells. This step is particularly advantageous and easy to carry out for secreted proteins that may be isolated simply from the cell culture supernatant. However, this step equally applies to isolating polypeptides from cellular membranes, or intracellular compartments. The process further comprises the step of purifying the proteins.

In yet a further preferred embodiment, the process further comprises the step of formulating the purified proteins into a pharmaceutical composition.

The invention further relates to an antibody specifically interacting with soluble IL-17RC of the invention, the antibody being preferably a monoclonal antibody, a polyclonal antibody, a humanized antibody or a human antibody.

A monoclonal antibody is an antibody composition having a homogeneous antibody population. The term is not limited regarding the species or source of the antibody. The term encompasses whole immunoglobulins as well as single chain antibodies. By contrast, polyclonal antibodies are antibodies that are derived from different cell lines and have specificity for different epitopes.

Methods of making polyclonal and monoclonal antibodies are known in the art. Polyclonal antibodies are generated by immunizing a suitable animal, such as a mouse, rat, rabbit, sheep, chicken, or goat, with an antigen of interest, such as a stem cell transformed with a gene encoding an antigen. In order to enhance immunogenicity, the antigen can be linked to a carrier prior to immunization. Suitable carriers are typically large, slowly metabolized macromolecules such as proteins, polysaccharides, polylactic acids, polyglycolic acids, polymeric amino acids, amino acid copolymers, lipid aggregates (such as oil droplets or liposomes), and inactive virus particles. Such carriers are well known to those of ordinary skill in the art. Furthermore, the antigen may be conjugated to a bacterial toxoid, such as toxoid from diphtheria, tetanus, cholera, etc., in order to enhance the immunogenicity thereof. Antibodies may also be generated by in vitro immunization, using methods known in the art. Polyclonal antisera is then obtained from the immunized animal.

Monoclonal antibodies are generally prepared using the method of Kohler and Milstein (Kohler and Milstein, 1975), or a modification thereof. Typically, a mouse or rat is immunized as described above. However, rather than bleeding the animal to extract serum, the spleen (and optionally several large lymph nodes) is removed and dissociated into single cells. If desired, the spleen cells may be screened (after removal of non-specifically adherent cells) by applying a cell suspension to a plate or well coated with the antigen. B-cells, expressing membrane-bound immunoglobulin specific for the antigen, will bind to the plate, and are not rinsed away with the rest of the suspension. Resulting B-cells, or all dissociated spleen cells, are then induced to fuse with myeloma cells to form hybridomas, and are cultured in a selective medium (e.g., hypoxanthine, aminopterin, thymidine (HAT) medium). The resulting hybridomas are plated by limiting dilution, and are assayed for the production of antibodies, which bind specifically to the immunizing antigen (and which do not bind to unrelated antigens). The selected monoclonal antibody-secreting hybridomas are then cultured either in vitro (e.g., in tissue culture bottles or hollow fiber reactors), or in vivo (e.g., as ascites in mice).

Human monoclonal antibodies are obtained by using human rather than murine hybridomas.

Humanized forms of non-human (e.g., murine) antibodies are chimeric immunoglobulins, immunoglobulin chains or fragments thereof (such as Fv, Fab, Fab′, F(ab′) 2 or other antigen-binding subsequences of antibodies) which contain minimal sequence derived from non-human immunoglobulin. Humanized antibodies include human immunoglobulins (recipient antibody) in which residues from a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin (Jones et al., 1986; Riechmann et al., 1988).

Methods for humanizing non-human antibodies are well known in the art. Generally, a humanized antibody has one or more amino acid residues introduced into it from a source which is non-human. These non-human amino acid residues are often referred to as “import” residues, which are typically taken from an “import” variable domain. Humanization can be essentially performed following the method of Winter and co-workers (Jones et al., 1986; Riechmann et al., 1988; Verhoeyen et al., 1988), by substituting rodent CDRs or CDR sequences for the corresponding sequences of a human antibody. Accordingly, such “humanized” antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

Human antibodies can also be produced using various techniques known in the art, including phage display libraries (Marks et al., 1991). The techniques of Boerner et al. are also available for the preparation of human monoclonal antibodies (Boerner et al., 1991). Similarly, human antibodies can be made by introducing of human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Fishwild et al., 1996; Marks et al., 1992.

Being a naturally secreted isoform, sIL-17RC has biological advantage over artificially secreted versions. This natural isoform may be biologically active as soluble decoy receptor. IL-17RC is potentially interacting with IL-17 family cytokines involved in inflammatory diseases. Therefore another aspect of the invention relates to the use of a sIL-17RC for the manufacture of a medicament for the treatment and/or prevention of a disease selected form the group consisting of autoimmune diseases, inflammatory disorders or neurological diseases.

The terms “treating” and “preventing”, as used herein, should be understood as preventing, inhibiting, attenuating, ameliorating or reversing one or more symptoms or cause(s) of neurologic disease, as well as symptoms, diseases or complications accompanying neurologic disease. When “treating” neurologic disease, the substances according to the invention are given after onset of the disease, “prevention” relates to administration of the substances before signs of disease can be noted in the patient.

The term “neurological disease”, as used herein encompasses all known neurologic diseases or disorders, or injuries of the CNS or PNS.

Neurologic diseases comprise disorders linked to dysfunction of the CNS or PNS, such as diseases related to neurotransmission, headache, trauma of the head, CNS infections, neuro-opthalmologic and cranial nerve disorders, function and dysfunction of the cerebral lobes disorders of movement, stupor and coma, demyelinating diseases, delirium and dementia, craniocervical junction abnormalities, seizure disorders, spinal cord disorders, sleep disorders, disorders of the peripheral nervous system, cerebrovascular disease, or muscular disorders. For definitions of these disorders, see e.g. The Merck Manual for Diagnosis and Therapy, Seventeenth Edition, published by Merck Research Laboratories, 1999.

Preferably, the neurological diseases of the invention are selected from the group consisting of traumatic nerve injury, stroke, demyelinating diseases of the CNS or PNS, neuropathies and neurodegenerative diseases.

Traumatic nerve injury may concern the PNS or the CNS, it may be brain or spinal cord trauma, including paraplegia, as described in the “background of the invention” above.

Stroke may be caused by hypoxia or by ischemia of the brain. It is also called cerebrovascular disease or accident.

Peripheral Neuropathy may be related to a syndrome of sensory loss, muscle weakness and atrophy, decreased deep tendon reflexes, and vasomotor symptoms, alone or in any combination. Neuropathy may affect a single nerve (mononeuropathy), two or more nerves in separate areas (multiple mononeuropathy), or many nerves simultaneously (polyneuropathy). The axon may be primarily affected (e.g. in diabetes mellitus, Lyme disease, or uremia or with toxic agents), or the myelin sheath or Schwann cell (e.g. in acute or chronic inflammatory polyneuropathy, leukodystrophies, or Guillain-Barré syndrome). Further neuropathies, which may be treated in accordance with the present invention, may e.g. be due to lead toxicity, dapsone use, tick bite, porphyria, or Guillain-Barré syndrome, and they may primarily affect motor fibers. Others, such as those due to dorsal root ganglionitis of cancer, leprosy, AIDS, diabetes mellitus, or chronic pyridoxine intoxication, may primarily affect the dorsal root ganglia or sensory fibers, producing sensory symptoms. Cranial nerves may also be involved, such as e.g. in Guillain-Barré syndrome, Lyme disease, diabetes mellitus, and diphtheria.

In a further preferred embodiment, the neurologic disorder is a demyelinating disease. Demyelinating diseases preferably comprise demyelinating conditions of the CNS, like acute disseminated encephalomyelitis (ADEM) and multiple sclerosis (MS), as well as demyelinating diseases of the peripheral nervous system (PNS). The latter comprise diseases such as chronic inflammatory demyelinating polyradiculoneuropathy (CIDP and acute, monophasic disorders, such as the inflammatory demyelinating polyradiculoneuropathy termed Guillain-Barré syndrome (GBS).

Any autoimmune and/or an inflammatory disorder is comprised by the present invention. Particular disorders comprised are: inflammatory bowel diseases, Crohn's disease, ulcerative colitis, collagenous colitis, lymphocytic colitis, diversion colitis irritable bowel syndrome, neuroinflammation including multiple sclerosis; Guillan Barré syndrome, chronic inflammatory polyneuropathy (CIPN), lung diseases including acute respiratory distress syndrome; joint and bone diseases including osteoarthritis and rheumatoid arthritis; liver diseases including liver fibrosis, cirrhosis and chronic liver disease; fibrotic diseases including, lupus, glomerulosclerosis, systemic sclerosis skin fibrosis, post-radiation fibrosis and cystic fibrosis; vascular pathologies including atherosclerosis, cardiomyopathy and myocardial infarction; restenosis; and degenerative diseases of the central nervous system including amyotrophic lateral sclerosis or inflammatory disorders of the skin including scleroderma, psoriasis or atopic dermatitis.

The following disorders are also comprised by the present invention: asthma, obstructive airway disease, and potentially osteo-arthritis.

Inflammation is the body's basic response to a variety of external or internal insults, such as infectious agents, physical injury, hypoxia, or disease processes in nearly any organ or tissue in the body. Inflammation entails the four well-known symptoms, namely redness, heat, tenderness/pain, and swelling. More specifically, inflammation involves assembly of immune system cells and molecules at a target site. Examples for chronic inflammatory diseases, are rheumatoid arthritis, inflammatory bowel disease, systemic lupus erythematosus, multiple sclerosis, and type 1 diabetes, for example. These diseases are also often characterized as autoimmune diseases or autoimmune/inflammatory disorders.

An autoimmune disease is a condition in which the body recognizes its own tissues as foreign and directs an immune response against them.

There are many different autoimmune diseases, and they can each affect the body in different ways. For example, the autoimmune reaction is directed against the brain in multiple sclerosis and the gut in Crohn's disease. In other autoimmune diseases such as systemic lupus erythematosus (lupus), affected tissues and organs may vary among individuals with the same disease. One person with lupus may have affected skin and joints whereas another may have affected skin, kidney, and lungs. Ultimately, damage to certain tissues by the immune system may be permanent, as with destruction of insulin-producing cells of the pancreas in Type 1 diabetes mellitus.

The invention further relates to the use of a nucleic acid molecule for manufacture of a medicament for the treatment and/or prevention of a neurologic disease, wherein the nucleic acid molecule comprises a nucleic acid sequence of SEQ ID NO: 2 or a nucleic acid sequence encoding a polypeptide comprising an amino acid sequence of SEQ ID NO: 1.

The invention further relates to a pharmaceutical composition comprising soluble IL-17RC, optionally together with one or more pharmaceutically acceptable excipients, for treatment and/or prevention an autoimmune and/or an inflammatory disorder and/or a neurological disease.

The definition of “pharmaceutically acceptable” is meant to encompass any carrier, which does not interfere with effectiveness of the biological activity of the active ingredient and that is not toxic to the host to which it is administered, or that can increase the activity. For example, for parenteral administration, the active protein(s) may be formulated in a unit dosage form for injection in vehicles such as saline, dextrose solution, serum albumin and Ringer's solution.

The active ingredients of the pharmaceutical composition according to the invention can be administered to an individual in a variety of ways. The routes of administration include intradermal, transdermal (e.g. in slow release formulations), intramuscular, intraperitoneal, intravenous, subcutaneous, oral, epidural, topical, intrathecal, rectal, and intranasal routes. Any other therapeutically efficacious route of administration can be used, for example absorption through epithelial or endothelial tissues or by gene therapy wherein a DNA molecule encoding the active agent is administered to the patient (e.g. via a vector), which causes the active agent to be expressed and secreted in vivo.

In addition, the protein(s) according to the invention can be administered together with other components of biologically active agents such as pharmaceutically acceptable surfactants, excipients, carriers, diluents and vehicles.

For parenteral (e.g. intravenous, subcutaneous, intramuscular) administration, the active protein(s) can be formulated as a solution, suspension, emulsion or lyophilised powder in association with a pharmaceutically acceptable parenteral vehicle (e.g. water, saline, dextrose solution) and additives that maintain isotonicity (e.g. mannitol) or chemical stability (e.g. preservatives and buffers). The formulation is sterilized by commonly used techniques.

The bioavailability of the active protein(s) according to the invention can also be ameliorated by using conjugation procedures which increase the half-life of the molecule in the human body, for example linking the molecule to polyethylenglycol (PEG), as described in the PCT Patent Application WO 92/13095.

The therapeutically effective amounts of the active protein(s) will be a function of many variables, including the type of protein, the affinity of the protein, any residual cytotoxic activity exhibited by the antagonists, the route of administration, the clinical condition of the patient (including the desirability of maintaining a non-toxic level of endogenous sIL-17RC activity).

A “therapeutically effective amount” is such that when administered, the sIL-17RC exerts a beneficial effect on the neurologic disease. The dosage administered, as single or multiple doses, to an individual will vary depending upon a variety of factors, including sIL-17RC pharmacokinetic properties, the route of administration, patient conditions and characteristics (sex, age, body weight, health, size), extent of symptoms, concurrent treatments, frequency of treatment and the effect desired.

As mentioned above, sIL-17RC can preferably be used in an amount of about 0.001 to 100 mg/kg of body weight, or about 0.01 to 10 mg/kg of body weight or about 9, 8, 7, 6, 5, 4, 3, 2 or 1 mg/kg of body weight or about 0.1 to 1 mg/kg of body weight.

The route of administration, which is preferred according to the invention is administration by subcutaneous route. Intramuscular administration is further preferred according to the invention.

In further preferred embodiments, sIL-17RC is administered daily or every other day.

The daily doses are usually given in divided doses or in sustained release form effective to obtain the desired results. Second or subsequent administrations can be performed at a dosage which is the same, less than or greater than the initial or previous dose administered to the individual.

According to the invention, sIL-17RC can be administered prophylactically or therapeutically to an individual prior to, simultaneously or sequentially with other therapeutic regimens or agents (e.g. multiple drug regimens), in a therapeutically effective amount, in particular with an interferon. Active agents that are administered simultaneously with other therapeutic agents can be administered in the same or different compositions.

All references cited herein, including journal articles or abstracts, published or unpublished U.S. or foreign patent application, issued U.S. or foreign patents or any other references, are entirely incorporated by reference herein, including all data, tables, figures and text presented in the cited references. Additionally, the entire contents of the references cited within the references cited herein are also entirely incorporated by reference.

Reference to known method steps, conventional methods steps, known methods or conventional methods is not any way an admission that any aspect, description or embodiment of the present invention is disclosed, taught or suggested in the relevant art.

The foregoing description of the specific embodiments will so fully reveal the general nature of the invention that others can, by applying knowledge within the skill of the art (including the contents of the references cited herein), readily modify and/or adapt for various application such specific embodiments, without undue experimentation, without departing from the general concept of the present invention. Therefore, such adaptations and modifications are intended to be within the meaning of a range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance presented herein, in combination with the knowledge of one of ordinary skill in the art.

Having now described the invention, it will be more readily understood by reference to the following examples that are provided by way of illustration and are not intended to be limiting of the present invention.

### EXAMPLES

### Example 1

### Identification and Cloning of sIL-17RC

The cDNA clone IL-17RC_I22 (plasmid 17998) was identified as the sequence Hs-PA-sub_GTC_P03_I22 in a non-public (in-house) cDNA library collection by sequence homology to the IL-17RC protein during BLAST search for full length IL-17RC receptor. The cDNA plasmid was subsequently retrieved from the subtracted human preadipocytes induced cDNA library (normalized custom cDNA library in pCMVSport6.1 vector) and complete sequence analysis revealed its unique splicing pattern. This novel splice variant of IL-17RC, which translates into a naturally secreted soluble isoform, was named sIL-17RC.

Cloning of sIL-17RC

hs PA cDNA libraries were generated from resting preadipocytes and preadipocytes induced with the following cocktail:

TNF1 ug/ml;

IFNg 1 ug/ml;

LPS 10 ug/ml;

PMA 5 ng/ml;

indomethacin 100 ug/ml;

cycloheximide 50 ug/ml.

The RNA was extracted from the cells 3 h post-treatment. The cDNA libraries were custom made at Invitrogen (Resgen). A subtracted library (Hs-PA-sub) was also generated from the induced library, using the control untreated library as the driver. The induced (Hs-PA-ind) and subtracted (Hs-PA-sub) libraries were subjected to sequencing to a depth of 4000 and 5000 sequences respectively.

Construction of Mammalian Cell Expression Vectors for sIL-17RC

Due to cDNA library construction constraints, the plasmid 17998 encoding sIL-17RC lacks 46 amino acids of the IL-17RC N-terminal region. Therefore, in order to reconstitute and clone the full length sIL-17RC, plasmid 17998 together with plasmid 17996 (containing the 5′ end of IL-17RC from exon 1 onwards) were used as PCR templates to generate pEAK12d and pDEST12.2 expression clones containing the sIL-17RC ORF sequence with a 3′ sequence encoding a tag using the Gateway™ cloning methodology (Invitrogen). Thus, the predicted N-terminus (the first 46 bases prediction at the 5′ end including start codon) was added by overlap PCR.

Generation of Gateway Compatible sIL-17RC ORF Fused to an in Frame Tag Sequence.

The first stage of the Gateway cloning process involves a three step PCR reaction which generates the ORF of sIL-17RC flanked at the 5′ end by an attB1 recombination site and Kozak sequence, and flanked at the 3′ end by a sequence encoding an in-frame tag, a stop codon and the attB2 recombination site (Gateway compatible cDNA). The 46 missing bases at the 5′ end of the sIL-17RC prediction were added by a PCR overlap reaction of two PCR products generated on one hand with the IL17RC_I22FL-G1F and IL17RC_I22R1b primers from the plasmid 17996, and on the other hand with the IL17RC_I22F2b and IL17RC_I22FL-G1R from the plasmid 17998.

The first PCR reaction (in a final volume of 50 μl) contains respectively: 1 μl (30 ng) of plasmid 17996, 1.5 μl dNTPs (10 mM), 10 μl of 10× Pfx polymerase buffer, 1 μl MgSO4 (50 mM), 1×PCRₓ Enhancer solution (Invitrogen), 0.5 μl each of gene specific primer (100 μM) (IL17RC_I22FL-G1F and IL17RC_I22R1b) and 0.5 μl Platinum Pfx DNA polymerase (Invitrogen). The PCR reaction was performed using an initial denaturing step of 95° C. for 5 min, followed by 30 cycles of 94° C. for 15 s; 55° C. for 30 and 68° C. for 1 min; and a holding cycle of 4° C. A 25 μl aliquot of the amplification product PCR1 was loaded on 0.8% agarose gel in 1×TAE buffer gel and the band at the expected molecular weight (322 bp) was purified using the Wizard SV Gel and PCR Clean-up System (Promega) and recovered in 50 μl sterile water according to the manufacturer's instructions.

The second PCR reaction (in a final volume of 50 μl) contains respectively: 1 μl (30 ng) of plasmid 17998, 1.5 μl dNTPs (10 mM), 10 μl of 10× Pfx polymerase buffer, 1 μl MgSO4 (50 mM), 1×PCRₓ Enhancer solution (Invitrogen), 0.5 μl each of gene specific primer (100 μM) (IL17RC_I22F2b and IL17RC_I22FL-G1R) and 0.5 μl Platinum Pfx DNA polymerase (Invitrogen). The PCR reaction was performed using an initial denaturing step of 95° C. for 5 min, followed by 30 cycles of 94° C. for 15 s; 55° C. for 30 s and 68° C. for 1 min; and a holding cycle of 4° C. A 25 ul aliquot of the amplification product PCR2 was loaded on 0.8% agarose gel in 1×TAE buffer gel and the band at the expected molecular weight (913 bp) was purified using the Wizard SV Gel and PCR Clean-up System (Promega) and recovered in 50 μl sterile water according to the manufacturer's instructions.

The third PCR reaction (in a final volume of 50 μl) contained 5 μl of each purified PCR1 and PCR2 product, 1.5 μl dNTPs (10 mM), 10 μl of 10× Pfx polymerase buffer, 1 μl MgSO₄ (50 mM), 1×PCR. Enhancer solution (Invitrogen), 0.5 μl of each Gateway conversion primer (100 μM) (GCP forward and GCP reverse) and 0.5 μl of Platinum Pfx DNA polymerase. The PCR reaction was performed using an initial denaturing step of 95° C. for 5 min, followed by 30 cycles of 94° C. for 15 s; 55° C. for 30 s and 68° C. for 1 min; and a holding cycle of 4° C. A 25 ul aliquot of the amplification product PCR3 was loaded on 0.8% agarose gel in 1×TAE buffer gel and the band at the expected molecular weight (1211 bp) was purified using the Wizard SV Gel and PCR Clean-up System (Promega) and recovered in 50 μl sterile water according to the manufacturer's instructions.

Subcloning of Gateway Compatible sIL-17RC ORF into Gateway Entry Vector pDONR221 and Expression Vectors pEAK12d and pDEST12.2

The second stage of the Gateway cloning process involved subcloning of the Gateway modified PCR product into the Gateway entry vector pDONR221 (Invitrogen) as follows: 5 μl of purified product from PCR3 were incubated with 1.5 μl pDONR221 (plasmid ID 13578) vector (0.1 μg/μl), 2 μl BP buffer and 1.5 μl of BP clonase enzyme mix (Invitrogen) in a final volume of 10 μl at RT for 1 h. The reaction was stopped by addition of proteinase K 1 μl (2 μg/μl) and incubated at 37° C. for a further 10 min. An aliquot of this reaction (1 μl) was used to transform E. coli DH10B cells by electroporation as follows: a 25 μl aliquot of DH10B electrocompetent cells (Invitrogen) was thawed on ice and 1 μl of the BP reaction mix was added. The mixture was transferred to a chilled 0.1 cm electroporation cuvette and the cells electroporated using a BioRad Gene-Pulser™ according to the manufacturer's recommended protocol. SOC media (0.5 ml) which had been pre-warmed to room temperature was added immediately after electroporation. The mixture was transferred to a 15 ml snap-cap tube and incubated, with shaking (220 rpm) for 1 h at 37° C. Aliquots of the transformation mixture (40 μl and 100 μl) were then plated on L-broth (LB) plates containing kanamycin (40 μg/ml) and incubated overnight at 37° C.

Plasmid miniprep DNA was prepared from 5 ml culture from 8 of the resultant colonies using a Qiaprep BioRobot 8000 system (Qiagen). Plasmid DNA (150-200 ng) was subjected to DNA sequencing with 21M13, IL17RC-435F and M13Rev primers as described above using the BigDyeTerminator system (Applied Biosystems cat. no. 4336919) according to the manufacturer's instructions. The primer sequences are shown in Table 1. Sequencing reactions were purified using Montage SEQ 96 cleanup plates (Millipore cat. no. LSKS09624) then analyzed on an Applied Biosystems 3700 sequencer.

Plasmid eluate (2 μl or approx. 150 ng) from one of the clones which contained the correct sequence (pENTR_sIL-17RC-tag, plasmid ID 18201) was then used in a recombination reaction containing 1.5 μl of either pEAK12d vector or pDEST12.2 vector (0.1 μg/μl), 2 μl LR buffer and 1.5 μl of LR clonase (Invitrogen) in a final volume of 10 μl. The mixture was incubated at RT for 1 h, stopped by addition of proteinase K (2 μg) and incubated at 37° C. for a further 10 min. An aliquot of this reaction (1 μl) was used to transform E. coli DH10B cells by electroporation as follows: a 25 μl aliquot of DH10B electrocompetent cells (Invitrogen) was thawed on ice and 1 μl of the LR reaction mix was added. The mixture was transferred to a chilled 0.1 cm electroporation cuvette and the cells electroporated using a BioRad Gene-Pulser™ according to the manufacturer's recommended protocol. SOC media (0.5 ml) which had been pre-warmed to room temperature was added immediately after electroporation. The mixture was transferred to a 15 ml snap-cap tube and incubated, with shaking (220 rpm) for 1 h at 37° C. Aliquots of the transformation mixture (10 μl and 50 μl) were then plated on L-broth (LB) plates containing ampicillin (100 μg/ml) and incubated overnight at 37° C.

Plasmid mini-prep DNA was prepared from 5 ml cultures from 6 of the resultant colonies subcloned in each vector using a Qiaprep BioRobot 8000 system (Qiagen). Plasmid DNA (200-500 ng) in the pEAK12d vector was subjected to DNA sequencing with pEAK12F, IL17RC-435F and pEAK12R primers as described above. Plasmid DNA (200-500 ng) in the pDEST12.2 vector was subjected to DNA sequencing with 21M13, IL17RC-435F and M13Rev primers as described above. Primer sequences are shown in Table 1.

CsCl gradient purified maxi-prep DNA was prepared from a 500 ml culture of the sequence verified clone (pEAK12d_sIL-17RC-tag, plasmid ID 18202) using the method described by Sambrook J. et al., 1989 (in Molecular Cloning, a Laboratory Manual, 2nd edition, Cold Spring Harbor Laboratory Press), Plasmid DNA was resuspended at a concentration of 1 μg/μl in sterile water (or 10 mM Tris-HCl pH 8.5) and stored at −20° C.

Endotoxin-free maxi-prep DNA was prepared from a 500 ml culture of the sequence verified clone (pDEST12.2_sIL-17RC-tag, plasmid ID 18203) using the EndoFree Plasmid Mega kit (Qiagen) according to the manufacturer's instructions. Purified plasmid DNA was resuspended in endotoxin free TE buffer at a final concentration of at least 3 μg/μl and stored at −20° C.

Site-directed PCR mutagenesis was performed to generate sIL-17RCexo7 expression constructs, used as controls in Example 2 below. The amino acid sequence of sIL-17RCexo7 (reported herein as SEQ ID NO: 3) differs by 1 aa (Arg instead of Gln in position 292) from a soluble splice variant protein #6 reported in WO 02/38764 as SEQ ID NO:15. For the generation of the sIL-17RCexo7 constructs, the DNA sequence coding for the 15 aa DCRGLEVWNSIPSCW corresponding to exon 7 of IL-17RC was inserted in the original sIL17-RC plasmid 17998 using the QuickChange XL site-directed mutagenesis Kit (Stratagene) and 74 mer oligonucleotides IL17RC_exon A and IL17RC_exon B according to manufacturer's instructions. Further cloning of full length sIL-17RCexo7 into pEAK12d (18283) and pDEST12.2 (18284) was performed as described above for sIL-17RC).

Table 1

| | |
|---|---|
| Primer | Sequence (5′-3′) |

| | |
|---|---|
| IL17RC_I22FL-G1F | GCA GGC TTC GCC ACC ATG CCT GTG |
| | CCC TGG TTC TT |
| IL17RC_I22R1b | C CTC ATC TTC AGG CTC TTC |
| IL17RC_I22F2b | AC TGG GAA GAG CCT GAA G |
| IL17RC_I22FL-G1R | TG ATG GTG ATG GTG TCG TCC CAT AGC |
| | TGC AGA C |
| GCP Forward | G GGG ACA AGT TTG TAC AAA AAA GCA |
| | GGC TTC GCC ACC |
| GCP Reverse | GGG GAC CAC TTT GTA CAA GAA AGC |
| | TGG GTT TCA ATG GTG ATG GTG ATG |
| | GTG |
| pEAK12F | GCC AGC TTG GCA CTT GAT GT |
| pEAK12R | GAT GGA GGT GGA CGT GTC AG |
| 21M13 | TGT AAA ACG ACG GCC AGT |
| M13REV | CAG GAA ACA GCT ATG ACC |
| IL17RC-435F | T GCT GCC CTT GTG CAG TTT G |
| IL17RC_exon A | CAC AGC AGC TGC CTG ACT GCA GGG |
| | GGC TCG AAG TCT GGA ACA GCA TCC |
| | CGA GCT GCT GGG CCC TGC CCT GGC |
| | TC |
| IL17RC_exon B | GAG CCA GGG CAG GGC CCA GCA GCT |
| | CGG GAT GCT GTT CCA GAC TTC GAG |
| | CCC CCT GCA GTC AGG CAG CTG CTG |
| | TG |

Underlined = Kozak sequence; Bold = Stop codon; Italic = tag

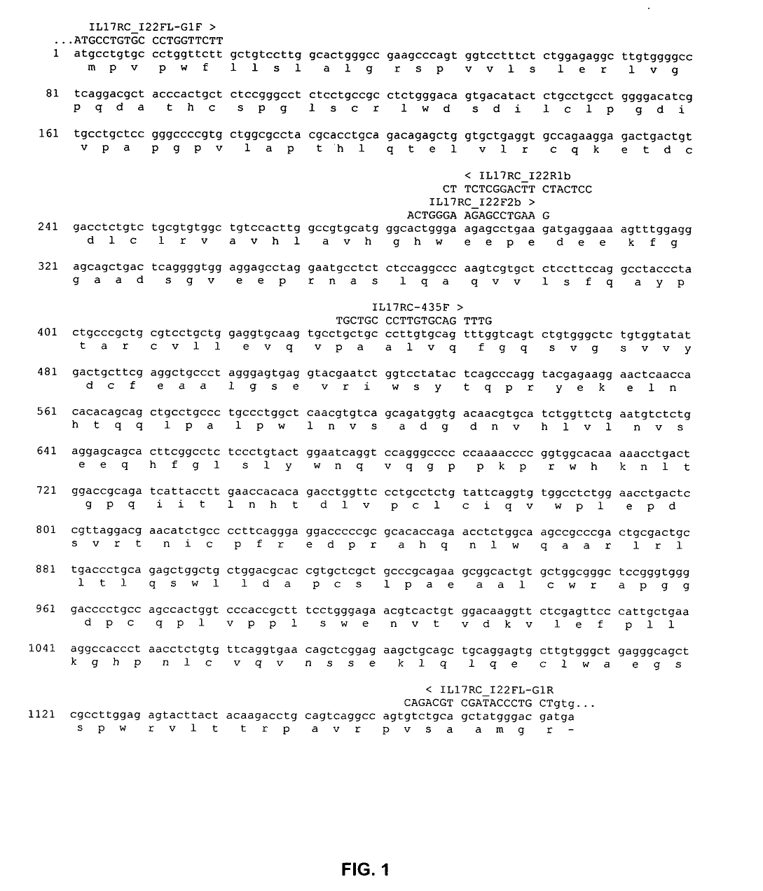

The alignment of sIL-17RC cDNA, protein and primer sequences is shown in FIG. 1.

### Example 2

### Secretion of sIL-17RC Form HEK293 Transfected Cells

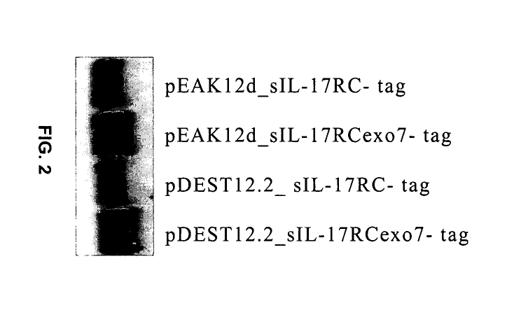

In order to verify that the novel sIL-17RC and control sIL-17RCexo7 isoforms are secreted, the corresponding pEAK12d and pDEST12.2 constructs were transfected into HEK293 cells using lipofectamine reagent (Invitrogen) according to manufacturer's instructions. 3 days after transfection, 20 ul of transfected cell supernatants were collected and separated on a 4-12% NuPage Bis-Tris gel with 1×MOPS SDS running buffer according to manufacturer's instructions (invitrogen). Efficient expression and secretion of sIL-17RC and sIL-17RCexo7 proteins was demonstrated using a mouse anti-tag antibody (QIAGEN) and anti-mouse HRP secondary antibody (Sigma) by Western blotting according to manufacturer's instructions (see FIG. 2).

### Example 3

### Inhibition of Ligand Binding Using sIL-17RC

For this assay, cell lines expressing endogenous or overexpressed exogenous receptors, such as a full length membrane bound human IL-17RC, are incubated with IL-17 family members, e.g. a human IL-17F tagged protein. Cells bound by the ligand are detected with an anti-tag antibody or an anti-IL-7F monoclonal antibody, followed by a secondary fluorescent-labeled antibody and measured by FACS (fluorescence activated cell sorting). The sIL-17RC protein is tested at different concentrations during the ligand binding incubation step for determining the inhibition of the binding activity. Inhibition of ligand binding is also verified using conventional in vitro binding techniques.

### Example 4

### Autoimmunity/Inflammatory Assays

The following assays can be used to confirm the biological activity of a sIL-17RC polypeptide.

### Assays Targeting T Lymphocyte Responses

Fas-Ligand-induced T cell death. This assay will reveal new modulators of receptor mediated cell death. In this assay, T cell apoptosis is induced by stimulating Jurkat cells (a human T cell line) with recombinant 6 Histidine-tagged Fas Ligand combined with a monoclonal anti tag antibody. Death is quantified by release of LDH, a cytoplasmic enzyme released in the culture medium when cells are dying. The read out is a colorimetric assay read at 490 nm.T cells have been shown to be pathogenic in many autoimmune diseases, being able to control antigen-specific T cell death is a therapeutic strategy (e.g. anti-TNFα treatment in patient with Crohn's disease).

Human-MLR: proliferation and cytokine secretion. This cell-based assay measures the effects of novel proteins on lymphocyte proliferation and cytokine secretion or inhibition upon stimulation by PBMC from another donor (alloreactivity). These assay address antigen-specific T cell and antigen presenting cell functions, which are crucial cellular responses in any autoimmune diseases. Secreted cytokine (IL-2, 4, 5, 10, TNF-a and IFN-g) are quantified by CBA.

Note: proliferation and cytokine secretion are independent responses.

Mouse-MLR: proliferation. This cell-based assay measures the effects of novel proteins on lymphocyte proliferation or inhibition of mouse spleen cells following stimulation by spleen cells from another donor (mouse strain). This cell-based assay measures the effect of novel proteins on T lymphocyte and antigen presenting cell responses and will be used to confirm activity of positives and hits identify in the h-MLR assays. This assay will be use to select proteins that will be tested in murine model of human diseases.

Human PBMC stimulated with the superantigen, TSST. Superantigens are strong modulators of the immune system affecting T cells. Superantigens influence immunologically mediated disorders such as IBD, inflammatory skin diseases like atopic dermatitis and psoriasis. In this cellular assay, we are specifically targeting T lymphocyte activation via the TCR but with different requirements than the T cell response to classical antigens, in particular in respect to co-stimulatory molecules.

Human PBMC stimulated with either ConA or PHA. These cell-based assays measure the effects of novel proteins on cytokine secretion induced by two different stimuli acting on different cells as measured by a cytokine bead array (CBA) assay (IL-2, IFN-g, TNF-a, IL-5, IL-4 and IL-10).

Most of cytokines can have dual actions, pro or anti-inflammatory, depending of the injury, milieu and cellular target. Any protein with the capability to modulate cytokine secretion may have a therapeutic potential (e.g. decreasing IFN-g and TNF-a would be beneficial in Th1-mediated autoimmune disease in contrast decreasing IL-4, IL-5 may be beneficial in Th2-mediated-diseases, inducing IL-10 would interesting in MS and SLE).

### Assays Targeting Monocyte/Macrophages and Granulocyte Responses

Human PBMC stimulated with LPS. This cell-based assay measures the effects of novel proteins on cytokine secretion (IFN-g, TNF-a) induced by LPS acting on monocytes/macrophages and granulocytes.

Any protein with the capability to modulate IFN-g and TNF-a secretion would be beneficial in Th1-mediated autoimmune diseases.

### Assays Targeting Neutrophil Responses

Neutrophils are important in inflammation and autoimmune diseases such as Rheumatoid Arthritis. Leukocyte chemo-attractants such as IL-8 initiate a sequence of adhesive interactions between cells and the micro-vascular endothelium, resulting in activation, adhesion and finally migration of neutrophils. The tissue infiltration of neutrophils depends on a reorganisation of cytoskeleton elements associated with specific changes in cell morphology of these cells.

This cell-based assay measures the effect of novel proteins on cytoskeleton reorganization of human neutrophils.

### Assays Targeting B Lymphocyte Responses

Autoantibodies as well as infiltrating B cells are thought to be important in the pathogenesis of various autoimmune diseases, such as systemic lupus erithematosus (SLE), rheumatoid arthritis (RA), Sjogren's syndrome and myasthenia gravis. Compelling evidence indicates that a disregulation in B cell homeostasis could affect immune tolerance leading to the inappropriate survival of autoreactive B cells producing pathogenic antibodies and sustained inflammation. The identification of new factors that play critical roles in the regulation of B cell proliferation, survival and differentiation following B cell receptor triggering are of high relevance in the development of novel therapies.

B cell proliferation. This cell-based assay measures the effect of novel proteins on B cell survival.

B cell co-stimulation. This cell-based assay measures the effect of novel proteins on B cell co-stimulation.

### Assays Targeting Monocytes and Microglial Responses

THP-1 calcium flux. The Ca+-flux in THP1-cell assay measures the effects of novel proteins on their ability to trigger an intracellular calcium release (a generic second messenger events) from the endoplasmic reticulum.

Microglia cell proliferation (will be Presented to the next IAC). During proliferation of microglial progenitors, a number of colony-stimulating factors, including some cytokines, are known to play key roles. Among them, M-CSF is crucial for the final step of maturation of macrophages/microglia and is not replaceable by any other factor. The evaluation of this biological response may represent a way to influence the microglial activity and therefore an opportunity to identify molecules with therapeutic potential for MS.

A cell-based assay was developed to measure the proliferative response of a microglia cell line to M-CSF. The feasibility and the robustness phases showed optimal results. This assay is in 96 well plates; non-radioactive substrate is required, easily automated.

### Example 5

### Neurological Assays

The following assays can be used to confirm the biological activity of an sIL-17RC polypeptide in neurological diseases. A number of neurological assays have been developed by the Applicant and are of use in the investigation of the biological relevance of protein function. Examples of neurological assays that have been developed by the Applicant include four types of assays. These are discussed below.

### Oligodendrocytes-Based Assays

Oligodendrocytes are responsible for myelin formation in the CNS. In multiple sclerosis they are the first cells attacked and their loss leads to major behavioral impairment. In addition to curbing inflammation, enhancing the incomplete remyelination of lesions that occurs in MS has been proposed as a therapeutic strategy for MS. Like neurons, mature oligodendrocytes do not divide but the new oligodendrocytes can arise from progenitors. There are very few of these progenitor cells in adult brain and even in embryos the number of progenitor cells is inadequate for HTS.

Oli-neu is a murine cell line obtained by an immortalization of an oligodendrocyte precursor by the t-neu oncogene. They are well studied and accepted as a representative cell line to study young oligodendrocyte biology. These cells can be used in two types of assays.

One, to identify factors stimulating oligodendrocytes proliferation, and the other to find factors promoting their differentiation. Both events are key in the perspective of helping renewal and repairing demyelinating diseases.

Another possible cell line is the human cell line, MO3-13. MO3-13 results from the fusion of rabdo-myosarcoma cells with adult human oligodendrocytes. However these cells have a reduced ability to differentiate into oligodendrocytes and their proliferating rate is not sufficient to allow a proliferation assay. Nevertheless, they express certain features of oligodendrocytes and their morphology is well adapted to nuclear translocation studies.

Therefore this cell line can be used in assays based on nuclear translocation of three transcription factors, respectively NF-kB, Stat-1 and Stat-2. The Jak/Stats transcription pathway is a complex pathway activated by many factors such as IFN a, b, g, cytokines (e.g. IL-2, IL-6; IL-5) or hormones (e.g. GH, TPO, EPO). The specificity of the response depends on the combination of activated Stats. For example, it is noticeable that IFN-b activates Stat1, 2 and 3 nuclear translocations meanwhile IFN-g only activates Stat1. In the same way, many cytokines and growth factors induced NF-kB translocation. In these assays the goal is to get a picture of activated pathways for a given protein.

### Astrocyte-Based Assays

The biology of astrocytes is very complex, but two general states are recognized. In one state called quiescent, astrocytes regulate the metabolic and excitatory level of neurons by pumping glutamate and providing energetic substratum to neurons and oligodendrocytes. In the activated state, astrocytes produce chemokines and cytokines as well as nitric oxide. The first state could be considered as normal healthy while the second state occurs during inflammation, stroke or neurodegenerative diseases. When this activated state persists it should be regarded as a pathological state.

Many factors and many pathways are known to modulate astrocyte activation. In order to identify new factors modulating astrocyte activation U373 cells, a human cell line of astroglioma origin, can be used. NF-kB, c-Jun as well as Stats are signaling molecules known to play pivotal roles in astrocyte activation.

A series of screens based on the nuclear translocation of NF-kB, c-Jun and Stat1, 2 and 3 can be carried out. Prototypical activators of these pathways are IL-1b, IFN-beta or IFN-gamma. The goal is to identify proteins that could be used as therapeutics in the treatment of CNS diseases.

### Neurons-Based Assays

Neurons are very complex and diverse cells but they have all in common two things. First they are post-mitotic cells, secondly they are innervating other cells. Their survival is linked to the presence of trophic factors often produced by the innervated target cells. In many neurodegenerative diseases the lost of target innervation leads to cell body atrophy and apoptotic cell death. Therefore identification of trophic factors supplementing target deficiency is very important in treatment of neurodegenerative diseases.

In this perspective a survival assay using NS1 cells, a sub-clone of rat PC12 cells, can be performed. These cells have been used for years and a lot of neurobiology knowledge has been first acquired on these cells before being confirmed on primary neurons including the pathways involved in neuron survival and differentiation (MEK, PI3K, CREB). In contrast the N2A cells, a mouse neuroblastoma, are not responding to classical neurotrophic factors but Jun-kinase inhibitors prevent apoptosis induced by serum deprivation. Therefore assays on these two cell lines will help to find different types of “surviving promoting” proteins.

It is important to note that in the previous assays we will identify factors that promote both proliferation and differentiation. In order to identify factors specifically promoting neuronal differentiation, a NS1 differentiation assay based on neurite outgrowth can be used. Promoting axonal or dendritic sprouting in neurodegenerative diseases could be advantageous for two reasons. It will first help the degenerating neurons to re-grow and re-establish a contact with the target cells. Secondly, it will potentiate the so-called collateral sprouting from healthy fibers, a compensatory phenomenon that delays terminal phases of neurodegenerative such as Parkinson or AD.

### Endothelial Cell-Based Assays

The blood brain barrier (BBB) between brain and vessels is responsible of differences between cortical spinal fluid and serum compositions. The BBB results from a tight contact between endothelial cells and astrocytes. It maintains an immunotolerant status by preventing leukocytes penetration in brain, and allows the development of two parallels endocrine systems using the same intracellular signaling pathways. However, in many diseases or traumas, the BBB integrity is altered and leukocytes as well as serum proteins enter the brain inducing neuroinflammation. There is no easy in vitro model of BBB, but cultures of primary endothelial cells such as human embryonic umbilical endothelial cells (HUVEC) could mimic some aspect of BBB biology. For example, BBB leakiness could be induced by proteins stimulating intracellular calcium release. In the perspective of identifying proteins that modulate BBB integrity, a calcium mobilization assay with or without thrombin can be performed on HUVEC.

### Example 6

### Mouse Models

The following mouse models can be used to confirm biological activity of a sIL-17RC polypeptide as disclosed herein. The mouse models are described in Chu et al. (Gene-engineered models for genetic manipulation and functional analysis of the cardiovascular system in mice. Chang Gung Med J. 2003 December; 26(12):868-78), in Ma et al. (Neurocardiovascular regulation in mice: experimental approaches and novel findings. Clin Exp Pharmacol Physiol. 2003 November; 30(11):885-93.) or Svenson et al. (Invited review: Identifying new mouse models of cardiovascular disease: a review of high-throughput screens of mutagenized and inbred strains. J Appl Physiol. 2003 April; 94(4):1650-9; discussion 1673).

Alternatively, the following mouse model can be used to confirm the biological activity of an sIL-17RC polypeptide as disclosed in Lu et al. (Lu et al. Nature. 2004 Nov. 11; 432(7014):179-86. Epub 2004 Oct. 27) by assaying sIL-17RC polypeptides in vascular development, specifically by measuring accumulation of blood in the venous circulation and fluid in the pericardial activity, or measuring peripheral resistance resulting from modified arterial vasculature, or measuring capillaries thickness and branching in hindbrains, or measuring filopodial extension from endothelial tip cells, or characterizing intersegmental blood vessels (ISVs) trajectory phenotypes and more generally vessel-branching defects.

### REFERENCE LIST

Boerner, P., Lafond, R., Lu, W. Z., Brams, P., and Royston, I. (1991). Production of antigen-specific human monoclonal antibodies from in vitro-primed human splenocytes. J Immunol 147, 86-95.
Fishwild, D. M., O'Donnell, S. L., Bengoechea, T., Hudson, D. V., Harding, F., Bernhard, S. L., Jones, D., Kay, R. M., Higgins, K. M., Schramm, S. R., and Lonberg, N. (1996). High-avidity human IgG kappa monoclonal antibodies from a novel strain of minilocus transgenic mice. Nat. Biotechnol. 14, 845-851.
Haudenschild, D., Moseley, T., Rose, L., and Reddi, A. H. (2002). Soluble and transmembrane isoforms of novel interleukin-17 receptor-like protein by RNA splicing and expression in prostate cancer. J Biol Chem 277, 4309-4316.
Hwang, S. Y. and Kim, H. Y. (2005). Expression of IL-17 homologs and their receptors in the synovial cells of rheumatoid arthritis patients. Mol Cells 19, 180-184.
Hymowitz, S. G., Filvaroff, E. H., Yin, J. P., Lee, J., Cai, L., Risser, P., Maruoka, M., Mao, W., Foster, J., Kelley, R. F., Pan, G., Gurney, A. L., de Vos, A. M., and Starovasnik, M. A. (2001). IL-17s adopt a cystine knot fold: structure and activity of a novel cytokine, IL-17F, and implications for receptor binding. EMBO J. 20, 5332-5341.
Jones, P. T., Dear, P. H., Foote, J., Neuberger, M. S., and Winter, G. (1986). Replacing the complementarity-determining regions in a human antibody with those from a mouse. Nature 321, 522-525.
Koenders, M. I., Lubberts, E., Oppers-Walgreen, B., van den, B. L., Helsen, M. M., Di Padova, F. E., Boots, A. M., Gram, H., Joosten, L. A., and van den Berg, W. B. (2005). Blocking of interleukin-17 during reactivation of experimental arthritis prevents joint inflammation and bone erosion by decreasing RANKL and interleukin-1. Am J. Pathol. 167, 141-149.
Kohler, G. and Milstein, C. (1975). Continuous cultures of fused cells secreting antibody of predefined specificity. Nature 256, 495-497.
Kolls, J. K. and Linden, A. (2004). Interleukin-17 family members and inflammation. Immunity. 21, 467-476.
Lee, J., Ho, W. H., Maruoka, M., Corpuz, R. T., Baldwin, D. T., Foster, J. S., Goddard, A. D., Yansura, D. G., Vandlen, R. L., Wood, W. I., and Gurney, A. L. (2001). IL-17E, a novel proinflammatory ligand for the IL-17 receptor homolog IL-17R1. J Biol Chem 276, 1660-1664.
Lubberts, E. (2003). The role of IL-17 and family members in the pathogenesis of arthritis. Curr Opin Investig. Drugs 4, 572-577.
Marks, J. D., Griffiths, A. D., Malmqvist, M., Clackson, T. P., Bye, J. M., and Winter, G. (1992). By-passing immunization: building high affinity human antibodies by chain shuffling. Biotechnology (N.Y.) 10, 779-783.
Marks, J. D., Hoogenboom, H. R., Bonnert, T. P., McCafferty, J., Griffiths, A. D., and Winter, G. (1991). By-passing immunization. Human antibodies from V-gene libraries displayed on phage. J Mol Biol 222, 581-597.
Moseley, T. A., Haudenschild, D. R., Rose, L., and Reddi, A. H. (2003). Interleukin-17 family and IL-17 receptors. Cytokine Growth Factor Rev 14, 155-174.
Quintavalla, J., Kumar, C., Daouti, S., Slosberg, E., and Uziel-Fusi, S. (2005). Chondrocyte cluster formation in agarose cultures as a functional assay to identify genes expressed in osteoarthritis. J Cell Physiol 204, 560-566.
Riechmann, L., Clark, M., Waldmann, H., and Winter, G. (1988). Reshaping human antibodies for therapy. Nature 332, 323-327.
Shi, Y., Ullrich, S. J., Zhang, J., Connolly, K., Grzegorzewski, K. J., Barber, M. C., Wang, W., Wathen, K., Hodge, V., Fisher, C. L., Olsen, H., Ruben, S. M., Knyazev, I., Cho, Y. H., Kao, V., Wilkinson, K. A., Carrell, J. A., and Ebner, R. (2000). A novel cytokine receptor-ligand pair. Identification, molecular characterization, and in vivo immunomodulatory activity. J Biol Chem 275, 19167-19176.
Tian, E., Sawyer, J. R., Largaespada, D. A., Jenkins, N. A., Copeland, N. G., and Shaughnessy, J. D., Jr. (2000). Evi27 encodes a novel membrane protein with homology to the IL17 receptor. Oncogene 19, 2098-2109.
Verhoeyen, M., Milstein, C., and Winter, G. (1988). Reshaping human antibodies: grafting an antilysozyme activity. Science 239, 1534-1536.
Yao, Z., Spriggs, M. K., Derry, J. M., Strockbine, L., Park, L. S., VandenBos, T., Zappone, J. D. Painter, S. L., and Armitage, R. J. (1997). Molecular characterization of the human interleukin (IL)-17 receptor. Cytokine 9, 794-800.
WO 02/38764
WO 2005/051422
WO01/04304

1-16. (canceled)

17. An isolated polypeptide comprising:
a) a soluble IL-17RC variant polypeptide comprising SEQ ID NO: 1;
b) a soluble IL-17RC variant polypeptide comprising SEQ ID NO: 1 fused to: a carrier molecule; peptide; a polypeptide; or a protein that promotes crossing of the blood brain barrier; or
c) a soluble PEGylated IL-17RC variant polypeptide comprising SEQ ID NO: 1.

18. The isolated polypeptide according to claim 17, wherein said polypeptide is a soluble IL-17RC variant polypeptide comprising SEQ ID NO: 1 that is fused to a carrier molecule; a peptide; a polypeptide; or a protein that promotes the crossing of the blood brain barrier.

19. The isolated polypeptide according to claim 17, wherein said polypeptide is a soluble PEGylated IL-17RC variant polypeptide.

20. The isolated polypeptide according to claim 17, wherein said soluble IL-17RC variant polypeptide comprising SEQ ID NO: 1 is fused to a polypeptide and said polypeptide is an immunoglobulin (Ig) molecule or the constant region of an immunoglobulin molecule.

21. The isolated polypeptide according to claim 17, wherein said soluble IL-17RC variant polypeptide comprises SEQ ID NO: 1.

22. An isolated nucleic acid molecule encoding:
a) a soluble IL-7RC variant polypeptide comprising SEQ ID NO: 1; or
b) a soluble IL-17RC variant polypeptide comprising SEQ ID NO: 1 fused to: a carrier molecule; peptide; a polypeptide; or a protein that promotes crossing of the blood brain barrier.

23. The isolated nucleic acid molecule of claim 22, wherein said nucleic acid molecule encodes a soluble IL-17RC variant polypeptide comprising SEQ ID NO: 1 that is fused to a carrier molecule; a peptide; a polypeptide; or a protein that promotes the crossing of the blood brain barrier.

24. The isolated nucleic acid molecule according to claim 22, wherein said nucleic acid molecule encodes a soluble PEGylated IL-17RC variant polypeptide comprising SEQ ID NO: 1.

25. The isolated nucleic acid molecule according to claim 22, wherein said nucleic acid molecule comprises SEQ ID NO: 2.

26. An expression vector comprising the nucleic acid molecule according to claim 22.

27. A host cell transfected with a vector according to claim 26.

28. A process for the production of a soluble IL-17RC variant proteins, comprising the step of culturing a host cell according to claim 27 under conditions that allow for the expression of said soluble IL-17RC variant proteins.

29. The process according to claim 28, further comprising isolating the expressed IL-17RC variant proteins from the host cell.

30. The process according to claim 28, further comprising purifying the expressed IL-17RC variant proteins.

31. The process according to claim 30, further comprising the step of formulating the purified IL-17RC variant proteins into a pharmaceutical composition.

32. An isolated antibody specifically binding with a soluble IL-17RC variant according to claim 17.

33. The isolated antibody according to claim 32, wherein said antibody is a monoclonal antibody, a polyclonal antibody, a humanized antibody or a human antibody.

34. A method of treating an autoimmune disease, inflammatory disorder or neurological disease comprising the administration of a composition comprising a polypeptide according to claim 17 to an individual having an autoimmune disease, inflammatory disorder or neurological disease in an amount effective to treat said disease or disorder.

35. A pharmaceutical composition comprising a soluble IL-17RC according to claim 17 and one or more pharmaceutically acceptable excipients.
